(19) United States
(12) Patent Application Publication
(10) Pub. No.: US 2004/0038272 A1
(43) Pub. Date: Feb. 26, 2004

(54) Microsatellite markers

(76) Inventors: Yuh-Shan Jou, Taipei (TW); Ya-Hui Chang, Tauchung (TW); Chuan-Chuan Chao, Taipei (TW)

Correspondence Address: FISH & RICHARDSON PC, 225 FRANKLIN ST, BOSTON, MA, 02110, US

(21) Appl. No.: 10/462,070

(22) Filed: Jun. 13, 2003

Related U.S. Application Data
(60) Provisional application No. 60/388,228, filed on Jun. 13, 2002.

(51) Int. Cl. C12Q 1/68; C07H 21/04

(52) U.S. Cl. 435/6; 536/23.1

(57) ABSTRACT

This invention features a marker set that includes different microsatellite markers corresponding respectively to different genetic loci, wherein a heterozygosity value for each genetic locus is at least 0.50 in the Mongoloid population, and the genetic distance between two adjacent microsatellite markers is in the average of 10 cM.

### RELATED APPLICATIONS

This application claims priority to U.S. Provisional Application Serial No. 60/388,228, filed Jun. 13, 2002, the contents of which are incorporated herein by reference.

### BACKGROUND

Microsatellites are tandemly repeated sequences of 2 to 6 base pairs (Tautz (1993) Exs. 67: 21-28). Although much remains unknown about their functions, a premise of using microsatellites as genetic markers is that their alleles vary only in the number of a repeat sequence (Guyer & Collins (1993) Am. J. Dis. Child. 147: 1145-1152). Microsatellite markers have been widely used as a powerful tool in genetic mapping (Roberts et al. (1999) Eur. J. Immunol. 29: 3047-3050), population genetics (Taylor et al. (1994) Mol. Ecol. 3: 277-290), linkage analysis (Georges et al. (1993) Proc. Natl. Acad. Sci USA 90: 1058-1062), evolutionary study (Bowcock et al. (1994) Nature 368: 455-457), and forensic medicine (Sacchetti et al. (1999) Clin Chem. 45: 178-183).

For example, microsatellite instability resulted from expansion or deletion of a repeated sequence has been detected in colorectal, endometrial, breast, gastric, pancreatic, and bladder neoplastic tissues. See, e.g., Risinger et al. (1993) Cancer Res. 53: 5100; Had et al. (1993) Cancer Res. 53: 5087; Peltomaki et al. (1993) Cancer Res. 53: 5853; and Gonzalez-Zulueta et al. (1993) Cancer Res. 53: 5620. Thus, these mutations can be used as specific markers for detection of cancer.

Microsatellites are known to have highly informative multialleles on a giving locus but vary among ethnic groups. Since high heterozygosity markers are crucial to reduce sample recruitment for effective and successful genotyping in a study cohort, there is a need to develop a set of microsatellites suitable for a particular population.

### SUMMARY

This invention relates to a microsatellite marker set that can be used to study the etiology of diseases and to test for individual identity and relationships in a Mongoloid population.

This invention features a marker set (i.e., a database) that includes different microsatellite markers corresponding respectively to different genetic loci, wherein a heterozygosity value for each genetic locus is at least 0.50 (e.g., any number between 0.50 and 1.00,) in a Mongoloid population (e.g., a Taiwanese population), and the genetic distance between two adjacent microsatellite markers averages 10 cM. The microsatellite markers and their corresponding genetic loci can be oligonucleotide repeats, such as di-nucleotide repeats, tri-nucleotide repeats, or tetra-nucleotide repeats. In some embodiments, at least 85% (e.g., any number between 85% and 100%,) of the microsatellite markers in this marker set are tri-nucleotide repeats or tetra-nucleotide repeats. In some embodiments, the genetic distance between two adjacent microsatellite markers is in the range of 1 to 35 cM.

An exemplary marker set of this invention includes at least 350 different microsatellite markers selected from Table 1 that correspond respectively to 350 different genetic loci. See the specific example below.

This invention also features a method for identifying a microsatellite marker that is related to a phenotype determined by genetic influence such as an inherited disease, a cancer or a human character of physical or psychological features (e.g. body height or pitch). The method includes (1) obtaining a nucleic acid from a patient that is from a Mongoloid population and suffers from the disease; (2) amplifying a segment of the nucleic acid, the segment, at least 50 nucleotides in length (e.g., 50 to 500 nucleotides), corresponding to a microsatellite marker in the above-described marker set; and (3) determining whether the amplified segment is different from an amplified segment acquired in the same manner from a healthy person. A statistic calculation of disease allele verse healthy allele is used in determination of significant association. Note that both the patient and the healthy person are from the same Mongoloid population. Further, step (3) can be performed by a size fractionation method (e.g., gel electrophoresis), by Mass spectrometry, or by any fragment sizing technologies to identify amplified segments.

As used herein, the term “microsatellite” refers to a tandem repeat sequence. A microsatellite can be represented by (X)ₙ, wherein X is an oligonucleotide (e.g., 2-6 bases in length), and n, the number of the repeat sequence, varies among ethnic groups. A “marker” is an identifier that corresponds to a unique sequence of a locus, e.g., presented as a pair of primers that can be used to amplify the unique sequence. A “microsatellite marker” is an identifier corresponding to a tandem repeat sequence that is genetically linked to a unique locus. A “locus” is the position on a chromosome. Different forms of alleles are found at the same locus. Note that in humans and other diploid organisms, except for sexual chromosomes, there are two alleles at the same locus, one on each chromosome of a parental chromosome pair. A “marker set,” as used herein, is a collection of microsatellite markers.

The term “heterozygosity value” refers to the proportion of heterozygous individuals at a genetic locus in a collection (e.g., 50% or 0.50), given the genotypes of all individuals in the collection. It can be calculated from the equation:

$$1 - {\sum }_{i = 1}^{n} {( f i )}^{2}$$

wherein fi is the allele frequency, and n is the total number of different alleles at the genetic locus.

The term “cM” is an abbreviation for centimorgan, which is a measure of genetic distance and indicates how far apart two genes (or loci) are. Generally, 1 cM equals about 1 million base pairs in human genome.

The term “Mongoloid” refers to humans featured by physical characteristics such as yellowish-brown skin pigmentation, straight black hair, dark eyes with pronounced epicanthic folds, and prominent cheekbones. The Mongoloid population includes peoples indigenous to central and eastern Asia, e.g., Chinese, Taiwanese, or Japanese.

As used herein, “an inherited disease” refers to a genetic disorder resulting from a defect in a gene or from a chromosomal abnormality. Examples of an inherited disease include, but are not limited to, coffin-lowry syndrome, cystic fibrosis, myotonic dystrophy, type 1 neurofibromatosis, Kennedy's disease, spinal bulbar muscular atrophy, types 1 and 3 spinocerebellar ataxia, coronary artery thrombosis, hemochromatosis, and diseases included in the OMIN database (see, e.g., http://www.ncbi.nlm.nih.gov/entrez/query.fcgi?db=OMIM). “Cancer” refers to cellular tumor. Cancer cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type, or stage of invasiveness. Examples of cancer include, but are not limited to, carcinoma and sarcoma such as leukemia, sarcomas, osteosarcoma, lymphomas, melanoma, ovarian cancer, skin cancer, testicular cancer, gastric cancer, pancreatic cancer, renal cancer, breast cancer, prostate colorectal cancer, cancer of head and neck, brain cancer, esophageal cancer, bladder cancer, adrenal cortical cancer, lung cancer, bronchus cancer, endometrial cancer, nasopharyngeal cancer, cervical or hepatic cancer, or cancer of unknown primary site.

Other features, objects, and advantages of the invention will be apparent from the description and from the claims.

### DETAILED DESCRIPTION

The present invention relates to a microsatellite marker set that contains different microsatellite markers, e.g., at least 350 different markers. The microsatellite marker set of this invention can be constructed by first obtaining microsatellite markers from public databases, and then selecting suitable microsatellite markers based on genetic studies on a Mongoloid population. Useful public databases include, but are not limited to, Genome database (GDB), Marshfield mapping center database, and the Cooperative Human Linkage Center (CHLC) database. See, e.g., Buetow et al. (1994) Nat. Genet. 6: 391-393, and Sheffield et al. (1995) Hum. Mol. Genet. 4: 1837-1844. These databases can be accessed from their on-line facilities using uniform resource locators that are well known to those skilled in the art. For example, microsatellite markers can be obtained from the CHLC database markers in version 8 Weber screening sets, which primarily contain tri- and tetra-nucleotide microsatellite markers. The markers are selected such that an interval genetic distance between two adjacent markers averages 10 cM. Each marker is then experimentally evaluated in a Mongoloid population and selected based on the heterozygosity value, i.e., at least 0.50.

More specifically, for each marker corresponding to a locus, the evaluation will be conducted by obtaining nucleic acids from a number of subjects from a Mongoloid population, amplifying segments of the nucleic acids with a pair of primers, identifying the amplified segments, determining whether the locus is heterozygous for each subject, and quantitating the heterozygosity value among all subjects. The sizes of the amplified segments are preferably in the range of 50 to 500 base pairs.

The sequence information of primers can be either retrieved from the databases described above or designed by a software program based on properties such as annealing temperature and internal pairing. Each pair of primers is used to amplify segments of a nucleic acid, e.g., by polymerase chain reaction (PCR). PCR can be carried out following standard procedures. For example, DNA is subjected to 35 cycles of amplification in a thermocycler as follows: 94° C. for 45 sec, 56° C. for 30 sec, and 72° C. for 1 min. After cycling amplification, a final extension step of 72° C. for 10 min will be conducted and stored at 12° C. To amplify nucleic acids from many loci obtained from the same individual, the nucleic acids can be multiplexed in a single amplification reaction by combining primers for more than one marker. See, e.g., Ausubel et al. (1989) Current Protocols in Molecular Biology John Wiley and Sons, New York; Innis et al. (1990) PCR Protocols: A Guide to Methods and Applications Academic Press, Harcourt Brace Javanovich, New York.

Identification of amplified segments of different sizes may be achieved using standard methods such as size fractionation, mass spectrometry-based detection or any fragment sizing technologies. Size fractionation separates DNA molecules according to their sizes, e.g., polyacrylamide gel electrophoresis. Size fractionation may also be accomplished by chromatographic methods known as gel filtration. The DNA segments in solution are separated according to their sizes as they pass through a column packed with a chromatographic gel. Mass spectrometry provides a means of “weighing” a DNA molecule by ionizing the molecule in vacuum and making it “fly” by volatilization. It can be used to simultaneously identify many DNA molecules. See, e.g., U.S. Pat. No. 6,268,144.

To facilitate the identification of amplified segments of different sizes, amplified segments can be labeled either during amplification, e.g., by the incorporation of labeled nucleotides, or using labeled primers. In addition to radioactive labels, other labels such as fluorescence, chemiluminescence, and electrochemical luminescence can be used. See Kricka (1992) Nonisotopic DNA Probe Techniques Academic Press, San Diego, pp. 3-28. Examples of fluorescent labels include fluoresceins, rhodamines (U.S. Pat. Nos. 5,366,860 and 5,936,087; 6,051,719), cyanines (U.S. Pat. No. 6,080,868 and WO 97/45539), and metal porphyrin complexes (WO 88/04777). In particular, fluorescence can be 6-carboxyfluorescein (FAM), 2′,4′,1,4,-tetrachlorofluorescein (TET), 2′,4′,5′,7′,1,4-hexachlorofluorescein (HEX; U.S. Pat. No. 5,654,442), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (U.S. Pat. Nos. 5,188,934 and 5,885,778), or 2′-chloro-7′-phenyl-1,4-dichloro-6-carboxyfluorescein 6 (U.S. Pat. No. 6,008,379). Rhodamine can be tetramethyl-6-carboxyrhodamine (TAMRA) or tetrapropano-6-carboxyrhodamine (ROX), and cyanine can be anthraquinone, malachite green, or a nitrothiazole or nitroimidazole compound.

Labeled amplified segments can be characterized directly by autoradiography or by laser detection, followed by computer assisted graphic display and analysis. For example, when different fluorescent labels are used, multiplexed or pooled PCR products can be analyzed simultaneously by using CCD camera, Genescan, and Genotyper softwares (Applied Biosystems). Genescan and Genotyper softwares can further manipulate the data by automatically inputting marker names from a data file and outputting them into data format of Excel or Text.

For each marker corresponding to a locus, once the amplified segments for each subject of different sizes have been identified, whether the locus in that subject is heterozygous can be determined. If a locus is heterozygous, two distinguishable alleles could be detected. The heterozygosity value among all subjects for this maker can be obtained. Any disqualified marker, i.e., having a heterozygosity value lower than 0.50, is eliminated.

This invention also provides a method to identify a disease-related locus and lead to identify a disease-associated gene. The method includes obtaining a nucleic acid from a patient; amplifying a segment of the nucleic acid; and determining whether the amplified segment is different from an amplified segment acquired in the same manner from a healthy person. The patient and the healthy person can be the same or different. For example, samples, from a patient suffering a cancer, obtained from tumor tissues and from the same person's normal tissues are amplified by a number of primer pairs, each pair corresponding to a microsatellite marker in the microsatellite marker set. For a microsatellite marker, if an amplified segment from nucleic acids from the tumor tissues is different from that from the normal tissues, this marker can be potentially identified as a cancer-related microsatellite marker. After amplification of the same marker on expanded samples from the same ethnic group with significant association by statistic analysis, such a marker can be used to screen for subjects having an increased risk of developing the cancer. In another example for linkage analysis or detection of disease susceptibility, samples from a patient suffering an inherited disease and from a healthy person are amplified by a number of primer pairs, each pair corresponding to a microsatellite marker in the microsatellite marker set. The patient and the healthy person may be individuals from the same family or the same population.

To determine whether an amplified segment from nucleic acids of a patient differs from that of a healthy person, standard methods such as size fractionation or mass spectrometry-based detection can be used. Alternatively, an amplified segment can be identified after being detected by an array, which contains a locus or marker specific oligonucleotide probes immobilized onto a substrate. The substrate has many addresses, and can be opaque, translucent, or transparent. The addresses can be distributed, on the substrate in one, two, or three dimensions. Examples of two-dimensional array substrates include glass slides, quartz, single crystal silicon, wafers, mass spectroscopy plates, metal-coated substrates, membranes, plastics and polymers (e.g., polystyrene, polypropylene or polyvinylidene difluoride). Three-dimensional array substrates include porous matrices, e.g., gels or matrices. Still other substrates include surfaces of microfluidic channels and devices, such as “Lab-On-A-Chip™” (Caliper Technologies Corp.). A locus or marker specific oligonucleotide array can be fabricated by a variety of methods, e.g., photolithographic methods (U.S. Pat. Nos. 5,143,854; 5,510,270; and. 5,527,681), mechanical methods (e.g., directed-flow methods as described in U.S. Pat. No. 5,384,261), pin based methods (U.S. Pat. No. 5,288,514), and bead based techniques (e.g., as described in PCT US/93/04145). Amplified segments hybridize to a probe array under proper hybridization conditions. Guidance for performing hybridization reactions can be found in Current Protocols in Molecular Biology, John Wiley & Sons, N.Y. (1989), 6.3.1-6.3.6. After introduction of labels by, e.g., a primer extension reaction, the array is detected to characterize bound amplified segments according to labels at addresses. Detection can be by image acquisition or other methods.

The microsatellite marker set of this invention can also be used in linkage analysis, loss of heterozygosity analysis, and forensic applications. See, e.g., Current Protocols in Human Genetics (2002) John Wiley and Sons, on line version.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications, including patents, recited herein are hereby incorporated by reference in their entirety.

### EXAMPLE

Genomic DNAs were obtained from 96 subjects randomly selected from a Taiwanese population. Microsatellite markers were first chosen from the CHLC database markers in version 8 Weber screening sets. For each marker, the sequence information of a pair of primers were retrieved from the CHLC or other public databases, and the genomic DNAs from each subject were amplified with the pair of primers containing fluorescence-labels, using the following PCR protocol: PCR of each DNA segment of interest was performed in a 96-well plate with a volume of 10 μL, containing 10 ng genomic DNA, 0.25 mM dNTPs, 0.3 pmol of each primer, 0.5 U of Taq polymerase (AmpliTaq Gold, AmpliTaq, or KlenTaq). After a pre-PCR heating step of 2 min at 94° C., 35 cycles of amplification (45 sec at 94° C. for denaturing, 30 sec at 56° C. for annealing, and 1 min at 72° C. for extension) were performed in a thermalcycler, followed by 10 min at 72° C. for final extension. PCR products were then pooled according to their fragments and fluorescent labels, separated and identified by laser detection, followed by computer assisted graphic display and analysis, Genescan and Genotyper softwares, respectively. Once the genomic DNAs from all subjects were amplified for all chosen markers and their PCR products were identified, a heterozygosity value was determined for each marker. A marker was retained if its heterozygosity value was at least 0.50. It was removed if its heterozygosity was lower than 0.50. Table 1 shows a microsatellite marker set thus constructed.

In Table 1, “locus name” represents a locus to which a microsatellite marker corresponds, “probe name” refers to a pair of primers used to amplify a segment at the locus, “Kosambi cM” refers to a genetic distance of the locus. The heterozygosity values, shown in Table 1, were experimentally determined in the manner as described above.

TABLE 1
A microsatellite marker set for a Taiwanese population.

| | | | | |
|---|---|---|---|---|
| | | Kosambi | | Variation |
| Locus Name | Probe Name | cM | Heterozygosity | Types |

Chromosome 1

| | | | | |
|---|---|---|---|---|
| D1S468 | AFM280we5 | 4.22 | 0.77 | 2n |
| D1S1612 | GGAA3A07 | 16.22 | 0.78 | 4n |
| D1S1151 | UT491 | 24.68 | 0.93 | 4n |
| D1S3669 | GATA29A05 | 37.05 | 0.71 | 4n |
| D1S3726 | ATA43C09 | 45.33 | 0.74 | 3n |
| | GGAA30B06 | 48.53 | 0.73 | 4n |
| D1S1676 | GGAA22F10 | 55.10 | 0.85 | 4n |
| | GATA137F01 | 64.38 | 0.76 | 4n |
| D1S3721 | GATA129H04 | 72.59 | 0.82 | 4n |
| D1S2134 | GATA72H07 | 75.66 | 0.72 | 4n |
| D1S3728 | GATA165C03 | 89.49 | 0.78 | 4n |
| D1S3467 | GATA28F10 | 97.49 | 0.75 | 4n |
| D1S1665 | GATA61A06 | 102.02 | 0.76 | 4n |
| D1S551 | GATA6A05 | 113.69 | 0.77 | 4n |
| D1S1658 | GATA45B07 | 131.34 | 0.73 | 4n |
| D1S1631 | ATA29D04 | 136.88 | 0.78 | 3n |
| D1S3723 | GATA176G01 | 140.39 | 0.83 | 4n |
| D1S534 | GATA12A07 | 151.88 | 0.78 | 4n |
| D1S1153 | UT666 | 161.05 | 0.90 | 4n |
| D1S1679 | GGAA5F09 | 170.84 | 0.84 | 4n |
| D1S318 | Mfd147 | 182.351 | 0.82 | 2n |
| D1S518 | GATA7C01 | 202.19 | 0.78 | 4n |
| D1S1660 | GATA48B01 | 212.44 | 0.80 | 4n |
| D1S3761 | GATA124F08 | 226.16 | 0.75 | 4n |
| D1S549 | GATA4H09 | 239.66 | 0.76 | 4n |
| D1S1644 | GATA23F09 | 242.34 | 0.70 | 4n |
| D1S2800 | AFMb360zg1 | 252.12 | 0.82 | 2n |
| D1S547 | GATA4A09 | 267.51 | 0.73 | 4n |
| D1S1609 | GATA50F11 | 274.53 | 0.84 | 4n |
| D1S2826 | AFM323zh1 | 285.75 | 0.71 | 2n |

Chromosome 2

| | | | | |
|---|---|---|---|---|
| D2S1780 | GATA72G11 | ˜10.3 | 0.70 | 4n |
| D2S2952 | GATA116B01 | 17.88 | 0.75 | 4n |
| D2S262 | UT595 | 27.60 | 0.81 | 4n |
| D2S272 | UT868 | 37.38 | 0.88 | 4n |
| D2S1788 | GATA86E02 | 55.51 | 0.87 | 4n |
| | GATA194B06 | 61.66 | 0.84 | 4n |
| D2S1352 | ATA27D04 | 73.61 | 0.70 | 3n |
| D2S1772 | GATA66D01 | 85.48 | 0.83 | 4n |
| D2S1387 | GATA62B10 | 103.16 | 0.70 | 4n |
| D2S1343 | ATA19E11 | 115.49 | 0.76 | 3n |
| D2S437 | GATA6A03 | 125.18 | 0.74 | 4n |
| D2S275 | UT5135 | 132.58 | 0.88 | 4n |
| D2S1334 | GATA4D07 | 145.08 | 0.91 | 4n |
| D2S442 | GATA8H05 | 147.40 | 0.76 | 4n |
| D2S1399 | GGAA20G04 | 152.04 | 0.85 | 4n |
| D2S142 | AFM191wg9 | 161.26 | 0.70 | 2n |
| D2S1776 | GATA71D01 | 173.00 | 0.72 | 4n |
| D2S1244 | UT500 | 182.56 | 0.88 | 4n |
| D2S1245 | | | | |
| D2S1361 | GATA14E05 | 188.11 | 0.75 | 4n |
| D2S2960 | | | | |
| D2S1384 | GATA52A04 | 200.43 | 0.79 | 4n |
| D2S2944 | GATA30E06 | 210.43 | 0.77 | 4n |
| D2S434 | GATA4G12 | 215.78 | 0.77 | 4n |
| D2S1363 | GATA23D03 | 227.00 | 0.76 | 4n |
| D2S1279 | UT8067 | 240.79 | 0.82 | 4n |
| D2S2973 | GATA151D12 | 247.85 | 0.73 | 4n |
| D2S125 | AFM112yd4 | 260.63 | 0.79 | 2n |

Chromosome 3

| | | | | |
|---|---|---|---|---|
| D3S1297 | AFM217xd2 | 8.31 | 0.72 | 2n |
| D3S3030 | GATA112H08 | ˜18.6 | 0.78 | 4n |
| D3S4545 | GATA164B08 | 26.25 | 0.74 | 4n |
| | ATA9B09 | 38.28 | 0.71 | 3n |
| D3S2466 | GGAA22H08 | 50.25 | 0.82 | 4n |
| D3S2432 | GATA27C08 | 57.92 | 0.81 | 4n |
| D3S1768 | GATA8B05 | 61.52 | 0.75 | 4n |
| D3S1766 | GATA6F06 | 78.64 | 0.71 | 4n |
| D3S4542 | GATA148E04 | 89.91 | 0.76 | 4n |
| D3S2454 | GATA52H09 | 97.75 | 0.76 | 4n |
| D3S2406 | GGAT2G03 | 102.64 | 0.91 | 4n |
| D3S4529 | GATA128C02 | 112.42 | 0.73 | 4n |
| D3S2459 | GATA68D03 | 119.09 | 0.82 | 4n |
| D3S3045 | GATA84B12 | 124.16 | 0.79 | 4n |
| D3S2460 | GATA68F07 | 134.64 | 0.76 | 4n |
| D3S1764 | GATA4A10 | 152.62 | 0.73 | 4n |
| D3S1744 | GATA3C02 | 161.04 | 0.80 | 4n |
| D3S2440 | | | | |
| D3S1763 | GATA3H01 | 176.54 | 0.72 | 4n |
| D3S2427 | GATA22F11 | 188.29 | 0.87 | 4n |
| D3S1754 | GATA14G12 | 190.43 | 0.73 | 4n |
| D3S2398 | GATA6G12 | 209.41 | 0.81 | 4n |
| D3S3054 | GGAA22B10 | 214.45 | 0.71 | 4n |
| D3S1311 | AFM254ve1 | 224.88 | 0.70 | 2n |

Chromosome 4

| | | | | |
|---|---|---|---|---|
| D4S2366 | GATA22G05 | 12.93 | 0.72 | 4n |
| D4S2639 | GATA90B10 | 33.42 | 0.81 | 4n |
| D4S2397 | ATA27C07 | 42.74 | 0.71 | 3n |
| D4S2632 | GATA72G09 | 50.53 | 0.87 | 4n |
| D4S1627 | GATA7D01 | 60.16 | 0.77 | 4n |
| D4S3254 | GATA61B02 | 63.58 | 0.79 | 4n |
| D4S3248 | GATA28F03 | 72.52 | 0.76 | 4n |
| D4S392 | AFM022xc1 | 78.97 | 0.76 | 2n |
| D4S3243 | GATA10G07 | 88.35 | 0.73 | 4n |
| D4S2409 | GATA26B12 | 96.16 | 0.77 | 4n |
| D4S1647 | GATA2F11 | 104.94 | 0.70 | 4n |
| D4S2623 | GATA62A12 | 114.04 | 0.83 | 4n |
| D4S3250 | GATA30B11 | 126.15 | 0.79 | 4n |
| D4S1625 | GATA107 | 145.98 | 0.70 | 4n |
| D4S1629 | GATA8A05 | 157.99 | 0.71 | 4n |
| D4S2414 | GATA30F07 | 167.55 | 0.83 | 4n |
| D4S2431 | GGAA19H07 | 176.19 | 0.80 | 4n |
| D4S2374 | GATA42E01 | ˜187.2 | 0.72 | 4n |
| D4S2930 | AFM224xh1 | 208.07 | 0.75 | 2n |

Chromosome 5

| D5S807 | GATA3A04 | 19.02 | 0.75 | 4n |
|---|---|---|---|---|
| D5S2845 | GATA134B03 | 36.25 | 0.78 | 4n |
| D5S1470 | GATA7C06 | 45.34 | 0.76 | 4n |
| D5S1506 | GATA63C02 | 49.54 | 0.72 | 4n |
| D5S1457 | GATA21D04 | 59.30 | 0.74 | 4n |
| D5S2507 | GGATA1D10 | 66.81 | 0.72 | 4n |
| D5S2500 | GATA67D03 | 69.23 | 0.77 | 4n |
| D5S806 | GATA5E10 | ˜84.0 | 0.76 | 4n |
| D5S1725 | GATA89G08 | 97.82 | 0.73 | 4n |
| D5S1462 | GATA3H06 | 105.29 | 0.79 | 4n |
| D5S1453 | ATA4D10 | 114.75 | 0.70 | 3n |
| D5S2501 | GATA68A03 | 116.98 | 0.71 | 4n |
| D5S1505 | GATA62A04 | 129.83 | 0.82 | 4n |
| D5S816 | GATA2H09 | 139.33 | 0.74 | 4n |
| D5S1469 | GATA51B01 | 152.62 | 0.80 | 4n |
| D5S820 | GATA6E05 | 159.77 | 0.75 | 3n |
| D5S422 | AFM211yc7 | 164.19 | 0.78 | 2n |
| D5S1456 | GATA11A11 | 174.80 | 0.76 | 4n |
| D5S408 | AFM164xb8 | 195.49 | 0.73 | 2n |
| | | Kosambi | | Variation |
| Locus Name | Marker | cM | Heterozygosity | Types |

Chromosome 6

| D6S344 | AFM092xb7 | 1.40 | 0.74 | 2n |
|---|---|---|---|---|
| D6S309 | AFM265zh9 | 14.07 | 0.80 | 2n |
| D6S2434 | ATA50C05 | 25.08 | 0.76 | 3n |
| D9S289 | AFM200wc9 | 29.93 | 0.81 | 2n |
| D6S2439 | GATA163B10 | 42.27 | 0.79 | 4n |
| D6S2427 | GGAA15B08 | 53.81 | 0.74 | 4n |
| D6S1017 | GGAT3H10 | 63.28 | 0.74 | 4n |
| D6S2410 | GATA11E02 | 73.13 | 0.71 | 4n |
| D6S1053 | GATA64D02 | 80.45 | 0.76 | 4n |
| D6S1609 | AFMb022xg9 | 92.25 | 0.75 | 2n |
| D6S1043 | GATA30A08 | 100.91 | 0.86 | 4n |
| D6S1284 | GGAA23B02 | 104.71 | 0.85 | 4n |
| D6S474 | GATA31 | 118.64 | 0.71 | 4n |
| D6S1958 | GATA28G05 | 125.71 | 0.74 | 4n |
| D6S1009 | GATA32B03 | 137.74 | 0.79 | 4n |
| | GATA184A08 | 146.06 | 0.82 | 4n |
| D6S2436 | GATA165G02 | 154.63 | 0.77 | 4n |
| D6S1035 | ATA6C09 | 164.78 | 0.70 | 3n |
| D6S1277 | GATA81B01 | 173.31 | 0.72 | 4n |
| D6S1027 | ATA22G07 | 187.23 | 0.70 | 3n |
| | | Kosambi | | Variation |
| Locus Name | Probe Name | cM | Heterozygosity | Types |

Chromosome 7

| | | | | |
|---|---|---|---|---|
| D7S517 | AFM225xa1 | 7.44 | 0.78 | 2n |
| D7S3047 | GATA119B03 | 17.17 | 0.72 | 4n |
| D7S2200 | | | | |
| D7S3051 | GATA137H02 | 29.28 | 0.79 | 4n |
| D7S1808 | GGAA3F06 | 41.69 | 0.75 | 4n |
| D7S817 | GATA13G11 | 50.29 | 0.73 | 4n |
| D7S1818 | GATA24D12 | 69.56 | 0.71 | 4n |
| D7S3046 | GATA118G10 | 78.65 | 0.83 | 4n |
| D7S1843 | GTAT1A10 | 83.99 | 0.80 | 4n |
| D7S3062 | | | | |
| D7S2204 | GATA73D10 | 90.95 | 0.80 | 4n |
| D7S820 | GATA3F01 | 98.44 | 0.72 | 4n |
| D7S1813 | ATA24A12 | 103.63 | 0.75 | 3n |
| D7S821 | GATA5D08 | 109.12 | 0.79 | 4n |
| D7S1799 | GATA23F05 | 113.92 | 0.72 | 4n |
| D7S1842 | GGAA6D03 | 128.41 | 0.77 | 4n |
| D7S1837 | GATA65F01 | ˜141.6 | 0.73 | 4n |
| D7S1824 | GATA32C12 | 149.90 | 0.71 | 4n |
| D7S2195 | GATA112F07 | 155.10 | 0.84 | 4n |
| D7S3070 | GATA189C06 | 163.03 | 0.80 | 4n |
| D7S3058 | GATA30D09 | 173.71 | 0.85 | 4n |
| D7S1823 | | | | |

Chromosome 8

| | | | | |
|---|---|---|---|---|
| D8S277 | AFM198wd2 | 8.34 | 0.81 | 2n |
| D8S1130 | GATA25C10 | 22.41 | 0.18 | 4n |
| D8S1145 | GATA72C10 | 37.04 | 0.79 | 4n |
| D8S322 | KW218 | 41.55 | 0.71 | 2n |
| D8S405 | UT5312 | | | |
| D8S382 | UT5185 | 51.15 | 0.76 | 4n |
| D8S1477 | GGAA20C10 | 60.34 | 0.77 | 4n |
| D8S1110 | GATA8G10 | 67.27 | 0.76 | 4n |
| D8S593 | GATA6F11 | ˜73.0 | 0.71 | 4n |
| D8S1136 | GATA41A01 | 82.26 | 0.72 | 4n |
| D8S2324 | GATA14E09 | 94.28 | 0.77 | 4n |
| D8S1119 | ATA19G07 | 101.01 | 0.73 | 3n |
| D8S1104 | GAAT1A4 | 110.20 | 0.72 | 4n |
| D8S1132 | GATA26E03 | 119.22 | 0.84 | 4n |
| D8S586 | GATA11E08 | 128.16 | 0.84 | 4n |
| D8S1179 | GATA7G07 | 135.08 | 0.83 | 4n |
| D8S1990 | GGAA23E06 | 150.51 | 0.75 | 4n |
| D8S373 | UT721 | 164.47 | 0.83 | 4n |

Chromosome 9

| | | | | |
|---|---|---|---|---|
| D9S288 | AEMa123xg1 | 9.83 | 0.86 | 2n |
| D9S2156 | GATA175H06 | 18.06 | 0.74 | 4n |
| D9S921 | GATA21A06 | 21.88 | 0.89 | 4n |
| D9S925 | GATA27A11 | 32.24 | 0.77 | 4n |
| D9S1121 | GATA87E02 | 44.28 | 0.79 | 4n |
| D9S1118 | GATA71E08 | 58.26 | 0.81 | 4n |
| D9S301 | GATA7D12 | 66.32 | 0.82 | 4n |
| D9S1122 | GATA89A11 | 75.88 | 0.70 | 4n |
| D9S922 | GATA21F05 | 80.31 | 0.71 | 4n |
| D9S283 | AFM318xc9 | 94.85 | 0.73 | 2n |
| D9S938 | GGAA22E01 | 110.93 | 0.76 | 4n |
| D9S930 | GATA48D07 | 120.04 | 0.78 | 4n |
| D9S934 | GATA64G07 | 127.98 | 0.77 | 4n |
| D9S1116 | GATA65D11 | 130.52 | 0.78 | 4n |
| D9S2152 | | | | |
| D9S752 | UT6068 | 141.69 | 0.76 | 4n |
| D9S2157 | ATA59H06 | 146.83 | 0.80 | 3n |
| D9S1826 | AFMb030zg9 | 159.61 | 0.82 | 2n |

Chromosome 10

| | | | | |
|---|---|---|---|---|
| D10S1435 | GATA88F09 | 4.32 | 0.71 | 4n |
| | ATA84D02 | 13.49 | 0.78 | 3n |
| D10S1216 | GGAA8G02 | 30.00 | 0.77 | 4n |
| D10S1430 | GATA84C01 | 33.18 | 0.79 | 4n |
| D10S1423 | GATA70E11 | 46.23 | 0.70 | 4n |
| D10S1426 | GATA73E11 | 59.03 | 0.76 | 4n |
| D10S1208 | ATA5A04 | 63.30 | 0.79 | 3n |
| D10S1221 | ATA21A03 | 75.57 | 0.78 | 3n |
| | GATA121A08 | 88.41 | 0.82 | 4n |
| D10S2327 | GGAT1A4 | 100.92 | 0.77 | 4n |
| D10S1427 | GATA81F06 | ˜104.0 | 0.79 | 4n |
| D10S1419 | GATA115E01 | 112.58 | 0.74 | 4n |
| D10S677 | GGAA2F11 | 117.42 | 0.77 | 4n |
| D10S521 | UT5027 | 127.11 | 0.75 | 4n |
| D10S1237 | GATA48G07 | 134.70 | 0.85 | 4n |
| D10S1230 | ATA29C03 | 142.78 | 0.73 | 3n |
| D10S217 | AFM212xd6 | 157.89 | 0.87 | 2n |
| D10S1248 | GGAA23C05 | 165.27 | 0.73 | 4n |

Chromosome 11

| | | | | |
|---|---|---|---|---|
| D11S2362 | ATA33B03 | 8.90 | 0.77 | 3n |
| D11S1997 | GATA13F08 | 12.92 | 0.77 | 4n |
| D11S4957 | | | | |
| D11S1981 | GATA48E02 | 21.47 | 0.79 | 4n |
| D11S904 | AFM081za5 | 33.57 | 0.72 | 2n |
| D11S1392 | GATA6B09 | 43.16 | 0.76 | 4n |
| D11S905 | AFM105xb10 | 51.95 | 0.81 | 2n |
| D11S987 | AFMa131ye5 | 67.48 | 0.82 | 2n |
| D11S2002 | GATA30G01 | 85.48 | 0.79 | 4n |
| D11S1367 | GATA7A03 | 90.89 | 0.76 | 4n |
| D11S1394 | GATA6C11 | 97.92 | 0.81 | 4n |
| D11S1986 | GGAA7G08 | 105.74 | 0.88 | 4n |
| D11S1998 | GATA23E06 | 113.13 | 0.78 | 4n |
| D11S4464 | GATA64D03 | 123.00 | 0.73 | 4n |
| D11S912 | AFM157xh6 | 131.26 | 0.82 | 2n |

Chromosome 12

| | | | | |
|---|---|---|---|---|
| D2S372 | GATA4H03 | 6.42 | 0.73 | 4n |
| D3S2395 | GATA49D12 | 17.72 | 0.71 | 4n |
| D12S391 | GATA11H08 | 26.23 | 0.85 | 4n |
| D12S373 | GATA6C01 | 36.06 | 0.79 | 4n |
| D12S1042 | ATA27A06 | 48.70 | 0.80 | 3n |
| D12S1301 | GATA91H06 | 56.25 | 0.71 | 4n |
| D12S390 | GATA11B02 | 67.63 | 0.71 | 4n |
| D12S1298 | GATA81H10 | 75.17 | 0.75 | 4n |
| D12S1052 | GATA26D02 | 83.19 | 0.76 | 4n |
| D12S1064 | GATA63D12 | 95.03 | 0.76 | 4n |
| D12S1300 | GATA85A04 | 104.12 | 0.70 | 4n |
| | PAH | 109.47 | 0.72 | 4n |
| | ATA63A05 | 1116.08 | 0.74 | 3n |
| D12S2070 | ATA25F09 | 125.31 | 0.73 | 3n |
| | PLA2 | 136.82 | 0.79 | 3n |
| D12S2078 | GATA32F05 | 149.60 | 0.77 | 4n |
| D12S1045 | ATA29A06 | 160.68 | 0.70 | 3n |

Chromosome 13

| | | | | |
|---|---|---|---|---|
| D13S742 | UT875 | 10.71 | 0.80 | 4n |
| D13S217 | AFM205xh12 | 17.21 | 0.77 | 2n |
| D13S1493 | GGAA29H03 | 25.80 | 0.77 | 4n |
| D13S325 | GATA6B07 | 38.96 | 0.80 | 4n |
| D13S1815 | GATA148B01 | 45.55 | 0.81 | 4n |
| D13S800 | GATA64F08 | 55.31 | 0.79 | 4n |
| D13S317 | GATA7G10 | 63.90 | 0.80 | 4n |
| D13S793 | GATA43H03 | 76.26 | 0.76 | 4n |
| D13S781 | ATA9E02 | 87.03 | 0.87 | 3n |
| D13S796 | GATA51B02 | 93.52 | 0.81 | 4n |
| D13S895 | GGAA22G01 | 98.82 | 0.72 | 4n |
| D13S285 | AFM309va9 | 110.55 | 0.85 | 2n |

Chromosome 14

| | | | | |
|---|---|---|---|---|
| D14S122 | UT1392 | 9.36 | 0.84 | 4n |
| D14S742 | GATA74E02 | 12.46 | 0.74 | 4n |
| D14S608 | GATA43H01 | 28.01 | 0.84 | 4n |
| D14S121 | UT1289 | 34.43 | 0.75 | 3n |
| D14S306 | GATA4B04 | 44.06 | 0.78 | 4n |
| D14S587 | GGAA10C09 | 55.82 | 0.85 | 4n |
| D14S592 | ATA19H08 | 66.81 | 0.74 | 3n |
| D14S588 | GGAA4A12 | 75.61 | 0.73 | 4n |
| D14S1433 | GATA169E06 | 84.69 | 0.79 | 4n |
| | GATA193A07 | 95.89 | 0.80 | 4n |
| D14S617 | GGAA21G11 | 105.53 | 0.79 | 4n |
| D14S1434 | GATA168F06 | 113.17 | 0.72 | 4n |
| D14S1426 | GATA136B01 | 125.88 | 0.76 | 4n |
| D14S292 | AFMa120xg5 | 134.30 | 0.70 | 2n |

Chromosome 15

| | | | | |
|---|---|---|---|---|
| D15S128 | AFM273yf9 | 6.11 | 0.84 | 2n |
| D15S822 | GATA88H02 | 12.30 | 0.82 | 4n |
| D15S1232 | GAAA1C11 | 31.46 | 0.85 | 4n |
| D15S659 | GATA63A03 | 43.47 | 0.84 | 4n |
| D15S643 | GATA50G06 | 52.33 | 0.85 | 4n |
| D15S153 | AFM205ye3 | 62.40 | 0.78 | 2n |
| D15S818 | GATA85D02 | 71.82 | 0.75 | 4n |
| D15S205 | AFM291zh5 | 78.92 | 0.86 | 2n |
| D15S652 | ATA24A08 | 90.02 | 0.86 | 3n |
| D15S816 | GATA73F01 | 100.28 | 0.73 | 4n |
| D15S657 | GATA22F01 | 104.86 | 0.83 | 4n |
| D15S642 | GATA27A03 | 122.14 | 0.76 | 4n |

Chromosome 16

| | | | | |
|---|---|---|---|---|
| D16S475 | UT581 | 7.61 | 0.85 | 4n |
| D16S2616 | ATA41E04 | 11.46 | 0.22 | 3n |
| D16S3075 | AFMb019zh9 | 23.28 | 0.82 | 2n |
| D16S3041 | AFM164th2 | 38.51 | 0.83 | 2n |
| D16S401 | AFM025tg9 | 46.94 | 0.73 | 2n |
| D16S753 | GGAA3G05 | 57.79 | 0.76 | 4n |
| D16S3396 | ATA55A11 | 63.78 | 0.77 | 3n |
| D16S2620 | GATA67G11 | 81.15 | 0.79 | 4n |
| D16S752 | GATA51G03 | 87.06 | 0.73 | 4n |
| D16S515 | AFM340ye5 | 92.10 | 0.84 | 2n |
| D16S511 | AFM312xd1 | 110.40 | 0.85 | 2n |
| D16S539 | GATA11C06 | 124.73 | 0.76 | 4n |
| D16S2621 | GATA71F09 | 130.41 | 0.71 | 4n |

Chromosome 17

| | | | | |
|---|---|---|---|---|
| | GATA158H04 | 14.69 | 0.75 | 4n |
| D17S974 | GATA8C04 | 22.24 | 0.73 | 4n |
| D17S900 | UT405 | 36.14 | 0.82 | 2n |
| D17S2196 | GATA185H04 | 44.62 | 0.78 | 4n |
| D17S1293 | GGAA7D11 | 56.33 | 0.84 | 4n |
| D17S1299 | GATA25A04 | 62.01 | 0.74 | 4n |
| D17S787 | AFM095tc5 | 74.99 | 0.81 | 2n |
| D17S1290 | GATA49C09 | 82.00 | 0.84 | 4n |
| D17S2193 | ATA43A10 | 89.32 | 0.75 | 3n |
| D17S949 | AFM292vh9 | 93.27 | 0.76 | 2n |
| D17S1862 | AFMc100c9 | 97.60 | 0.84 | 2n |
| D17S785 | AFM049xc1 | 103.53 | 0.73 | 2n |
| D17S1847 | AFMb310yf5 | 111.22 | 0.77 | 2n |
| D17S928 | AFM217yd10 | 126.46 | 0.82 | 2n |

Chromosome 18

| | | | | |
|---|---|---|---|---|
| | GATA178F11 | 2.84 | 0.81 | 4n |
| D18S1370 | ATA45G06 | 6.94 | 0.74 | 3n |
| D18S452 | AFM206xf4 | 18.70 | 0.80 | 2n |
| D18S542 | GATA11A06 | 41.24 | 0.80 | 4n |
| D18S869 | GATA41G05 | 49.55 | 0.75 | 4n |
| D18S535 | GATA13 | 64.48 | 0.78 | 4n |
| D18S851 | GATA6D09 | 74.93 | 0.75 | 4n |
| D18S1357 | ATA7D07 | 88.62 | 0.84 | 3n |
| D18S862 | | | | |
| D18S1364 | GATA7E12 | 99.04 | 0.83 | 4n |
| D18S878 | | | | |
| | ATA82B02 | 106.81 | 0.82 | 3n |
| D18S1362 | GATA51E05 | 109.18 | 0.73 | 4n |
| D18S870 | | | | |
| D18S844 | ATA1H06 | 116.44 | 0.78 | 3n |
| D18S70 | AFM254vd5 | 126.00 | 0.77 | 2n |

Chromosome 19

| | | | | |
|---|---|---|---|---|
| D19S591 | GATA44F10 | 9.84 | 0.71 | 4n |
| D19S592 | GATA47D11 | ˜24.1 | 0.83 | 4n |
| D19S1165 | GATA134B01 | 36.22 | 0.72 | 4n |
| D19S714 | GATA66B04 | 42.28 | 0.83 | 4n |
| D19S1037 | GGAA21A04 | 47.67 | 0.73 | 4n |
| D19S433 | GGAA2A03 | 51.88 | 0.75 | 4n |
| D19S718 | GATA84G04 | 65.77 | 0.85 | 4n |
| D19S541 | UT910 | ˜73.8 | 0.83 | 4n |
| D19S601 | GAAA1B03 | 83.19 | 0.76 | 4n |
| D19S589 | GATA29B01 | 87.66 | 0.78 | 4n |
| D19S544 | UT1342 | 100.01 | 0.79 | 4n |

Chromosome 20

| | | | | |
|---|---|---|---|---|
| D20S482 | GATA51D03 | 12.12 | 0.72 | 4n |
| D20S603 | GATA74A11 | ˜17.0 | 0.74 | 4n |
| D20S604 | GATA81E09 | 32.94 | 0.73 | 4n |
| D20S470 | GGAA7E02 | 39.25 | 0.87 | 4n |
| D20S477 | GATA29F06 | 47.52 | 0.79 | 4n |
| D20S478 | GATA42A03 | 54.09 | 0.80 | 4n |
| D20S481 | GATA47F05 | 62.32 | 0.72 | 4n |
| D20S159 | UT1307 | 69.50 | 0.83 | 4n |
| D20S480 | GATA45B10 | 79.91 | 0.80 | 4n |
| D20S171 | AFM046xf6 | 95.70 | 0.79 | 2n |

Chromosome 21

| | | | | |
|---|---|---|---|---|
| D21S1432 | GATA11C12 | 2.99 | 0.73 | 4n |
| D21S1436 | GGAA2E02 | 13.05 | 0.73 | 4n |
| D21S2052 | GATA129D11 | 24.73 | 0.82 | 4n |
| D21S1252 | AFM261zg1 | 35.45 | 0.79 | 2n |
| D21S2055 | GATA188F04 | 40.49 | 0.88 | 4n |
| D21S266 | AFM234cg9 | 45.87 | 0.85 | 2n |
| D21S1446 | GATA70B08 | 57.77 | 0.70 | 4n |

Chromosome 22

| | | | | |
|---|---|---|---|---|
| | GATA198B05 | 1.79 | 0.86 | 4n |
| D22S686 | GGAA10F06 | 13.60 | 0.70 | 4n |
| D22S690 | GATA46E03 | ˜23.2 | 0.86 | 4n |
| D22S689 | GATA21F03 | 28.57 | 0.80 | 4n |
| D22S683 | GATA11B12 | 36.22 | 0.86 | 4n |
| D22S417 | UT1091 | 46.42 | 0.85 | 4n |
| D22S274 | AFM164th8 | 51.54 | 0.76 | 2n |

Chromosome X

| | | | | |
|---|---|---|---|---|
| DXS9895 | GATA124B04 | 15.66 | 0.72 | 4n |
| DXS987 | AFM120xa9 | 22.18 | 0.79 | 2n |
| DXS9896 | GATA124E07 | 30.84 | 0.82 | 4n |
| DXS1214 | AFM283wg9 | 33.54 | 0.81 | 2n |
| DXS7132 | GATA72E05 | 52.50 | 0.78 | 4n |
| DXS6789 | GATA31F01 | 62.52 | 0.76 | 4n |
| | GATA172D05 | 68.74 | 0.76 | 4n |
| | GATA198A10 | 79.19 | 0.75 | 4n |
| DXS2390 | GATA31E08 | 87.56 | 0.77 | 4n |
| DXS8043 | AFMb018wd9 | 94.22 | 0.76 | 2n |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A marker set comprising different microsatellite markers corresponding respectively to different genetic loci, wherein a heterozygosity value for each genetic locus is at least 0.50 in a Mongoloid population, and the genetic distance between two adjacent microsatellite markers averages 10 cM.

2. The marker set of claim 1, wherein the heterozygosity value is at least 0.60.

3. The marker set of claim 2, wherein the heterozygosity value is at least 0.70.

4. The marker set of claim 1, wherein the microsatellite markers correspond to genetic loci that are oligonucleotide repeats.

5. The marker set of claim 4, wherein the heterozygosity value is at least 0.60.

6. The marker set of claim 5, wherein the heterozygosity value is at least 0.70.

7. The marker set of claim 4, wherein the genetic loci that are di-nucleotide repeats, tri-nucleotide repeats, or tetra-nucleotide repeats.

8. The marker set of claim 7, wherein the heterozygosity value is at least 0.60.

9. The marker set of claim 8, wherein the heterozygosity value is at least 0.70.

10. The method of claim 4, wherein at least 85% of the microsatellite markers correspond to genetic loci that are tri-nucleotide repeats or tetra-nucleotide repeats.

11. The method of claim 10, wherein at least 90% of the microsatellite markers correspond to genetic loci that are tri-nucleotide repeats or tetra-nucleotide repeats.

12. The marker set of claim 1, wherein the genetic distance between two adjacent microsatellite markers is in the range of 1 to 35 cM.

13. The marker set of claim 12, wherein the microsatellite markers correspond to genetic loci that are oligonucleotide repeats.

14. The marker set of claim 13, wherein the heterozygosity value is at least 0.60.

15. The marker set of claim 14, wherein the heterozygosity value is at least 0.70.

16. The marker set of claim 13, wherein the genetic loci that are di-nucleotide repeats, tri-nucleotide repeats, or tetra-nucleotide repeats.

17. The marker set of claim 16, wherein the heterozygosity value is at least 0.60.

18. The marker set of claim 17, wherein the heterozygosity value is at least 0.70.

19. The marker set of claim 1, wherein the Mongoloid population is a Taiwanese population.

20. A method for identifying a microsatellite marker related to a disease, comprising:
obtaining a nucleic acid from a patient that is from a Mongoloid population and suffers from the disease,
amplifying a segment of the nucleic acid, the segment corresponding to a microsatellite marker in a marker set that comprises different microsatellite markers corresponding respectively to different genetic loci, wherein a heterozygosity value for each genetic locus is at least 0.50 in the Mongoloid population, and the genetic distance between two adjacent microsatellite markers averages 10 cM, and
determining whether the amplified segment is different from an amplified segment acquired in the same manner from a healthy person from the Mongoloid population,
wherein a difference indicates that the microsatellite marker relates to the disease.

21. The method of claim 20, wherein the disease is an inherited disease.

22. The method of claim 21, wherein the heterozygosity value is at least 0.60.

23. The method of claim 22, wherein the heterozygosity value is at least 0.70.

24. The marker set of claim 21, wherein the microsatellite markers correspond to genetic loci that are oligonucleotide repeats.

25. The method of claim 23, wherein the heterozygosity value is at least 0.60.

26. The method of claim 25, wherein the heterozygosity value is at least 0.70.

27. The marker set of claim 24, wherein the genetic loci that are di-nucleotide repeats, tri-nucleotide repeats, or tetra-nucleotide repeats

28. The method of claim 27, wherein the heterozygosity value is at least 0.60.

29. The method of claim 28, wherein the heterozygosity value is at least 0.70.

30. The method of claim 24, wherein at least 85% of the microsatellite markers correspond to genetic loci that are tri-nucleotide repeats or tetra-nucleotide repeats.

31. The method of claim 31, wherein at least 90% of the microsatellite markers correspond to genetic loci that are tri-nucleotide repeats or tetra-nucleotide repeats.

32. The method of claim 21, wherein the genetic distance between two adjacent microsatellite markers is in the range of 1 to 35 cM.

33. The marker set of claim 32, wherein the microsatellite markers correspond to genetic loci that are oligonucleotide repeats.

34. The marker set of claim 33, wherein the genetic loci that are di-nucleotide repeats, tri-nucleotide repeats, or tetra-nucleotide repeats

35. The method of claim 21, wherein the Mongoloid population is a Taiwanese population.

36. The method of claim 20, wherein the patient has cancer.

37. The method of claim 36, wherein the heterozygosity value is at least 0.60.

38. The method of claim 37, wherein the heterozygosity value is at least 0.70.

39. The marker set of claim 36, wherein the microsatellite markers correspond to genetic loci that are oligonucleotide repeats.

40. The method of claim 39, wherein the heterozygosity value is at least 0.60

41. The method of claim 40, wherein the heterozygosity value is at least 0.70

42. The marker set of claim 39, wherein the genetic loci that are di-nucleotide repeats, tri-nucleotide repeats, or tetra-nucleotide repeats

43. The method of claim 42, wherein the heterozygosity value is at least 0.60.

44. The method of claim 43, wherein the heterozygosity value is at least 0.70.

45. The method of claim 39, wherein at least 85% of the microsatellite markers correspond to genetic loci that are tri-nucleotide repeats or tetra-nucleotide repeats.

46. The method of claim 45, wherein at least 90% of the microsatellite markers correspond to genetic loci that are tri-nucleotide repeats or tetra-nucleotide repeats.

47. The method of claim 36, wherein the genetic distance between two adjacent microsatellite markers is in the range of 1 to 35 cM.

48. The marker set of claim 47, wherein the microsatellite markers correspond to genetic loci that are oligonucleotide repeats.

49. The marker set of claim 48, wherein the genetic loci that are di-nucleotide repeats, tri-nucleotide repeats, or tetra-nucleotide repeats

50. The method of claim 36, wherein the Mongoloid population is a Taiwanese population.
